(19) United States
(12) Patent Application Publication
(10) Pub. No.: US 2024/0167014 A1
(43) Pub. Date: May 23, 2024

(54) Stable storage of enzymes

(71) Applicants: NEXTKIDNEY S.A., Lausanne (CH); Dialyss Pte Ltd, Singapore (SG)

(72) Inventors: Harmen Henri Jacobus De Jongh, Zeist (NL); Jacobus Adrianus Wilhelmus Jong, Utrecht (NL); Christian Gert Bluechel, Singapore (SG)

(73) Assignees: NEXTKIDNEY S.A., Lausanne (CH); Dialyss Pte Ltd, Singapore (SG)

(21) Appl. No.: 18/548,909

(22) PCT Filed: Mar. 4, 2022
(86) PCT No.: PCT/EP2022/055502

§ 371 Date: Sep. 4, 2023

(30) Foreign Application Priority Data
Mar. 5, 2021 (EP) 21161048.0

(51) Int. Cl. C12N 9/80; C12N 9/96

(52) U.S. Cl. CPC C12N 9/80; C12N 9/96; C12Y 305/01005

(57) ABSTRACT

The present invention relates to methods and compositions that are useful for improving the stability of an enzyme, for instance during storage. Using the methods and compositions of the invention, enzyme activity is preserved over time, allowing longer storage.

### FIELD OF THE INVENTION

The present invention relates to methods and compositions that are useful for improving the stability of an enzyme, for instance during storage. Using the methods and compositions of the invention, enzyme activity is preserved over time, allowing longer storage.

### BACKGROUND ART

Patients with end stage kidney disease (ESKD) or severe acute kidney failure can undergo dialysis (either hemodialysis, or HD, or peritoneal dialysis, or PD) to replace kidney function. Conventional dialysis is time-consuming, and removal of waste molecules and excess water is inadequate, contributing significantly to poor quality of life, severe health problems and high mortality (15-20% per year). Treatment costs are very high.

In conventional dialysis, patient fluids are generally dialysed against a dialysis fluid (referred to as dialysate), which is then discarded. During this process waste solutes from the patient fluid move towards the dialysate by diffusion and/or convection, often through a membrane such as a semipermeable membrane. This “single pass” use of dialysate has significant implications on infrastructural requirements, and cost of the therapies, as well as on the size and portability of the dialysis machines. It is thus desirable to reduce the volume of dialysis fluid. In miniaturisation efforts, patient fluids are dialysed against a relatively small amount of dialysis fluid, which is repeatedly regenerated and reused by removal of waste solutes from used dialysate. Efficient regeneration of dialysate would reduce the need for large volumes of dialysis fluid, making dialysis more practically implemented, less resource-dependent, and reducing waste streams.

A miniature artificial kidney device will be a major breakthrough in renal replacement therapy. Worldwide the number of dialysis patients is projected to reach 4.9 million by 2025. Currently, approximately 85% of the dialysis patients use HD techniques, either in a center (>96%) or at home (<4%). While in-center HD requires long frequent visits to the hospital (about 3 times per week, 4 h per session), home HD offers more flexibility and autonomy. However, today's home HD still requires bulky dialysis machines, which either require a large supply of dialysis fluids (at least 100 L per week) or must be connected to a bulky immobile water purification system. A user-friendly lightweight HD device that is independent of a fixed water supply or large supply of dialysis fluids will increase patients' mobility allowing them to stay active in social life and travel freely. It will further allow the patient to conduct more frequent dialysis in the comfort of their homes.

The large fluctuations in water balance and uremic toxin levels between dialysis treatments with standard thrice weekly HD could be attenuated with continuous or more frequent HD, which may improve patient outcome. A more liberal diet would be allowed. Significant cost reductions will be achieved through reduced need of dialysis personnel and related infrastructure, fewer medication and less hospitalizations due to reduced comorbidity.

PD is currently used by approximately 15% of the dialysis patients. Also this technique would significantly benefit from a miniaturised PD device that continuously regenerates the dialysate, thereby greatly enhancing PD efficacy. By preventing the two major causes of technique failure in conventional PD (recurrent infection and functional loss of the peritoneal membrane) a miniaturised PD device would further significantly prolong technique survival.

A user-friendly wearable or portable dialysis device, providing dialysis outside the hospital, would thus represent a huge leap forward for dialysis patients and would significantly increase their quality of life. The device would allow continuous or more frequent dialysis which will improve removal of waste solutes and excess fluid, and hence patient health. A miniaturized design, independent of a fixed water supply, offers freedom and autonomy to the patient.

In recent years, small prototype dialysis devices have been constructed that adequately remove some organic waste solutes and waste ions. However, thus far no adequate strategy for removal of urea exists, while urea is one of the main obstacles for successful realization of a miniature artificial kidney device. Urea is the waste solute with the highest daily production (as primary waste product of nitrogen metabolism) and exerts toxic effects at high plasma concentrations. However, urea is difficult to bind and has low reactivity.

EP121275A1/U.S. Pat. No. 4,897,200A discloses a ninhydrin-type sorbent that is formed out of a polymerized styrene composition in a six-step synthetic sequence. A urea binding capacity of 1.2 mmol/g dry sorbent in 8 hours was shown at clinically relevant urea concentrations. However, for effective miniaturisation, a higher urea binding capacity is required.

WO2017116515A1 discloses the use of electrically charged membranes to improve urea separation from a dialysis fluid, and suggests the use of electrooxidation of separated urea. A disadvantage of this method is that reactive oxygen species are generated as a byproduct.

WO2011102807A1 discloses epoxide-covered substrates. The epoxides can be used to recover solutes from a solution. They are also used to immobilise urease enzymes, which help dispose of urea. WO2016126596 also uses a very different substrate, viz. reduced graphene oxide. While a high urea binding capacity was shown, the captured urea represented less than 15% of the initial urea concentration.

A critical element in enzyme production and later use is to warrant the specific activity during prolonged storage. The preservation of the enzymatic activity can be affected by structural stability of the enzyme storage, by storage temperature (enzyme structural changes due to denaturation), by pH (extreme pH-values can denature the enzyme, and catalytic sites on the enzyme can be sensitive to the degree of protonation of acidic or basic groups). Enzyme stability can also be influenced by oxidation that may be irreversible and that may affect the chemical state of a number of the amino acid side groups, which may lead to reduced enzyme activity.

Among the 20 amino acids most commonly found in enzymes, several can be oxidised. Most susceptible amino acids are those having sulfhydryl groups (cysteine, methionine) and those with aromatic side chain groups (tryptophan, tyrosine, phenylalanine). Furthermore, histidine residues can be oxidised to 2-oxohistidine and 4-OH-glutamate, though tyrosine residues are converted to a dihydroxy-derivative, dopamine (DOPA), nitrotyrosine, chlorotyrosin and a dityrosine derivative. Finally, carbonyl groups can further react with amino groups of lysine residues, which lead to the formation of intra- or inter-molecular cross-links promoted protein aggregation (V. Cecarini et al., DOI:10.1016/j.bbamcr.2006.08.039).

Relationships between co-solutes such as salts, amino acids, carbohydrates, and protein structure or stability are well described, but not fully understood. Especially the use of carbohydrates or polyols has been promoted to preserve protein structural integrity over a broader range of external conditions, like temperature, pH or concentration, by affecting the thermodynamic state of the molecule (Van Teeffelen et al., Prot. Sci. 14, 2005; 2187-1294).

Enzymes are often stored in the presence of glucose to preserve functional characteristics during frozen storage or drying. To control oxidation, an extensive toolbox is available to preserve the desired reduced state of enzymes (for instance storage under inert atmosphere, or presence of antioxidants). For enzymes that are relevant for dialysis, such as urease, prolonged storage under known conditions was not found to be suitable (see e.g. FIG. 1). For urease storage, low temperature storage is recommended (DOI: 10.34049/bcc.51.2.4536).

To enable the development of improved artificial kidney devices, there is an ongoing need for improved means of urea removal, or for means that are more robust, that have a longer shelf life, or that can be stored under more different conditions such as at room temperature. There is a need for storable enzyme compositions that are stable under conditions of sterilization.

### SUMMARY OF THE INVENTION

The present invention provides methods and compositions that prolong the shelf life of enzymes, particularly of hydrolases such as urease. It was found that oligosaccharides helpfully increase shelf life stability. Accordingly the invention provides a method for improving the stability of an enzyme, comprising the steps of: i) providing an enzyme; ii) contacting the enzyme with a storage solution comprising an oligosaccharide to obtain a storage composition, and iii) optionally drying the storage composition. The enzyme can be a hydrolase, preferably an amidohydrolase, more preferably a urease. Preferably the enzyme has an active site comprising a nickel center, more preferably two nickel centers, even more preferably a bis-μ-ligand dimeric nickel center. The storage solution preferably further comprises buffer salts, antioxidants, bacteriostatics, chelators, cryo-protective agents, or serum albumins. Preferably the storage solution is buffered at a pH in the range of 5.5-8.2, and/or the storage solution is a pharmaceutically acceptable solution. Preferably the storage solution comprises 5-40 wt.-% of the oligosaccharide, more preferably 10-35 wt.-%, even more preferably 15-30 wt.-%. Sometimes the storage solution comprises about 25 wt.-% of the oligosaccharide and optionally buffer salts, preferably phosphate buffer salts. Preferably the oligosaccharide is an oligohexose, more preferably an oligoketohexose or an oligoaldohexose, even more preferably an oligoketohexose. Preferably the oligosaccharide has a degree of polymerisation of 2-75, more preferably of 2-20. Preferably the enzyme is an immobilized enzyme. Preferably the storage composition is stored for at least 25 days, wherein the enzyme retains at least 75% of its original activity after the storage.

Also provided is a composition comprising an enzyme as defined above and an oligosaccharide as defined above. Preferably the enzyme is an amidohydrolase, more preferably a urease, and/or the oligosaccharide is an oligohexose, preferably an oligoketohexose or an oligoaldohexose, more preferably an oligoketohexose. Also provided is a cartridge for use in a dialysis device, comprising such a composition.

Also provided is a method for storing an enzyme, the method comprising the steps of: I) providing a composition according to the invention, or a cartridge according to the invention, and II) storing the composition or cartridge for at least 2 days.

### DESCRIPTION OF EMBODIMENTS

The present invention provides methods and compositions that prolong the shelf life of enzymes, particularly of hydrolases such as urease. In a first aspect the invention provides a method for improving the stability of an enzyme, comprising the steps of:
i) providing an enzyme;
ii) contacting the enzyme with a storage solution comprising an oligosaccharide to obtain a storage composition, and
iii) optionally drying the storage composition.

Such a method is referred to hereinafter as a stabilizing method according to the invention. Preferably the steps are performed in numerical order. Preferably the dried storage composition is a homogenous mixture, a heterogeneous mixture, or surface coating of the storage solution on enzyme particles.

Step i)—Provision of an Enzyme

Enzymes can be obtained from any source, such as from commercial suppliers, from fermentation, or from isolation. The enzyme can be provided as a dry powder, as a solution, or as a suspension. Preferably the enzyme is substantially pure, or at least 80 wt.-% of proteinaceous material consists of the enzyme, more preferably at least 90%, most preferably at least 95% or even 99%. In other embodiments, less pure enzyme is used, which can be advantageous to for instance reduce manufacturing cost. Here, purity can be as low as 1, 2, 3, 4, 5, 6, 7, 8, 9, or 10 wt.-%, such as about 10 wt.-%. For example, purity can effectively be between 1-99 wt.-%, preferably between 10-90 wt.-%. When in a solution, the solvent is preferably water or acetic acid, most preferably water. For known enzymes, a skilled person is able to determine whether buffer salts are advantageous to use. The enzyme can also be immobilized on a solid support, such as on a resin, a polymer such as a bio-polymer, or a bead. A preferred enzyme is an enzyme that is sensitive to oxidation. In preferred embodiments the enzyme is immobilised as later described herein. In some embodiments the enzyme is preferably dry, or at least substantially dry, which herein should be understood as having a water content of at most 20 wt.-%, wherein a water content of about 10-15 wt.-% is particularly preferred for convenience of handling. In embodiments wherein the enzyme is immobilised, the enzyme can be either wet or dry. A wet immobilised enzyme can be preferred because it requires less processing steps because drying can be omitted.

The stabilization method according to the invention was found to produce attractive results for enzymes that feature particular oxidation-sensitive moieties, particularly in their active sites. Surprisingly, these enzymes could be stabilized without requiring the presence of antioxidants. Such enzymes preferably have an active site comprising a nickel center, preferably two nickel centers, more preferably a bis-μ-ligand dimeric nickel center. A μ-ligand is a bridging ligand. Such an active site preferably comprises two nickel centers that have a distance of about 3 to 4 Å. For instance, the active site of ureases is located in the α (alpha) subunits, and is a bis-μ-hydroxo dimeric nickel center, with an interatomic distance of ˜3.5 Å. Preferably, the nickel is Ni(II). Preferably, the two nickel atoms are weakly antiferromagnetically coupled. X-ray absorption spectroscopy (XAS) studies of urease from various sources (Canavalia ensiformis (jack bean), Klebsiella aerogenes and Sporosarcina pasteurii) confirm 5-6 coordinate nickel ions that exclusively have O/N ligation, including two imidazole ligands per nickel.

It was found that the stabilizing method according to the invention is particularly useful for stabilizing hydrolase enzymes. Hydrolases are a class of enzymes classified as EC 3, which generally use water to break a chemical bond. Suitable hydrolases are esterases, phosphatases, glycosidases, peptidases, nucleosidases, ureohydrolases, and amidohydrolases.

Hydrolases inherently have degradative properties and therefore their stabilisation is of particular utility. Preferred hydrolases are hydrolases that cleave non-peptide carbon-nitrogen bonds (classified as EC 3.5). Of these, amidohydrolases (EC 3.5.1 for linear amides and EC 3.5.2 for cyclic amides) and ureohydrolases (EC 3.5.3) are of particular interest, where amidohydrolases for linear amides are most preferred. Examples of amidohydrolases for linear amides are asparaginase, glutaminase, urease, biotinidase, aspartoacylase, ceramidase, aspartylglucosaminidase, fatty acid amide hydrolase, and histone deacetylase. Preferred examples are urease and histone deacetylase, where urease is most preferred. Accordingly, in preferred embodiments the enzyme is a hydrolase, preferably an amidohydrolase, more preferably a urease.

Urease, also known as urea amidohydrolase, catalyzes the hydrolysis of urea into carbon dioxide and ammonia. It is an enzyme found in numerous bacteria, fungi, algae, plants, and some invertebrates, as well as in soils, as a soil enzyme. Ureases are nickel-containing metalloenzymes of generally high molecular weight. The enzyme is most commonly assembled as trimers and hexamers of subunits with a molecular weight of about 90 kDa. Preferred ureases are urease from Canavalia ensiformis (jack bean), Glycine max, Oryza sativa, Cryptococcus neoformans, Arabidopsis thaliana, Yersinia pseudotuberculosis, Yersinia pestis, Rhizobium meliloti, Rhodopseudomonas palustris, Delftia acidovorans, Streptococcus thermophilus, Klebsiella aerogenes, Sporosarcina pasteurii, Helicobacter pylori, or Mycobacterium tuberculosis. Ureases from plant sources can be preferred for their ease of isolation from cultivated plants. Jack bean (Canavalia ensiformis) urease is highly preferred. In general, ureases are widely available from commercial suppliers.

Jack bean urease has two structural and one catalytic subunit. It is composed of 840 amino acids per monomer, assembling into the hexamer that is the active enzyme. There are 90 cysteine residues in the active enzyme. The molecular mass (without Ni(II) ions) is about 90.8 kDa. The mass of the hexamer including a total of 12 ligated nickel ions (2 per active site) is about 545 kDa. Accordingly, preferred enzymes for use in a stabilization method according to the invention are enzymes comprising a polypeptide having at least 50 amino acids, comprising at least 0.5% cysteine residues; more preferably comprising a polypeptide having at least 100 amino acids, even more preferably at least 200 amino acids, still more preferably at least 500 amino acids. The comprised cysteine residues are more preferably at least 1%, even more preferably at least 1.5%, most preferably at least 2%. These percentages refer to the percentage of amino acid residues. Most preferably the enzyme is multimeric. Preferably such a multimeric active enzyme has at least 1000 amino acids in its active enzyme, more preferably at least 2000, even more preferably at least 3000, most preferably at least 4000, such as at least 5000.

The enzyme can be stabilized while it is a free enzyme that is not linked to any other moiety or carrier. It can also be an immobilized enzyme. In preferred embodiments, the enzyme is a free enzyme, which is useful for subsequent applications in fully dissolved media, or in homogeneous catalysis. In preferred embodiments the enzyme is an immobilized enzyme, which is useful for subsequent applications in for instance in fixed-bed reactors, cartridges, or cassettes or columns, or heterogeneous catalysis. Immobilisation of enzymes is well known in the art, and enzymes provided in this step can be immobilised using known methods, for instance those described by v. Gelder et al, 2020, Biomaterials 234, 119735, or WO2011102807 or U.S. Pat. No. 8,561,811 or WO2016126596, or Zhang et al., DOI: 10.1021/acsomega.8b03287. Preferred for this invention are immobilized enzymes, particularly immobilized urease, wherein the enzyme is immobilized on a cellulose carrier, for instance as described in WO2011102807. The enzyme is preferably immobilized via covalent bonds, such as via amine, amide, or ether bonds, most preferably via amine or ether bonds. Such enzymes immobilized on a cellulose carrier are suitable for heterogeneous catalysis, for instance in a cartridge. Preferably, the immobilisation has already been performed prior to step i), in that the provided enzyme is already an immobilized enzyme. In preferred embodiments, the provided enzyme is of a single type, in that it is not a mixture of different enzymes.

Step ii)—Storage Solution

In step ii) the enzyme provided in step i) is contacted with a storage solution comprising an oligosaccharide to obtain a storage composition. Contacting can be achieved by any means. If the provided enzyme is in a dry state, it can be dissolved or suspended in the storage solution. If the provided enzyme is in a solution or suspension, it can be mixed with the storage solution, resulting in a dissolved or suspended enzyme also. If the enzyme is on a solid support, it can be submerged or suspended in the storage solution, or wetted with the storage solution. In particular embodiments when the enzyme is on a solid support, it is wetted with the storage solution. Preferably, the enzyme is dissolved or suspended in the storage solution. A skilled person will understand that if the enzyme is mixed with the storage solution from a dissolved state, the concentration of solutes in the storage solution is preferably adapted to the increase in volume, to achieve concentrations as described herein after the enzyme is admixed.

Conveniently, the dry components of the storage solution can be added to the provided enzyme, after which water is added to bring the storage composition to its desired volume. In preferred embodiments the storage solution is formed in situ, or in other words the storage solution is formed in the presence of the enzyme. Here, step ii) comprises a step ii-a): admixing the provided enzyme with the dry components of the storage solution, and ii-b) adding water to the mixture of ii-a) to obtain a storage composition.

The two most important components of the storage solution are water and an oligosaccharide. The water is preferably deionized water, pyrogen free water, water for injection, or ultrapure water. Preferably the amount of enzyme in the storage solution is in the range of 1 to 150 mg/mL, more preferably about 5 to 100, even more preferably about 10 to 50, still more preferably about 15 to 45, most preferably about 20 to 40, such as about 30.

Oligosaccharides are well known and are widely available from commercial sources. Oligosaccharides can also be isolated from natural sources such as from plants, for instance from chicory, which is a useful source of inulin. Preferably, oligosaccharides are used as such, and are not linked to further moieties such as lipids or peptides.

Polysaccharides are known to have degrees of polymerisation that can be up to 3000, i.e. comprise up to and including 3000 monomers. An oligosaccharide is generally shorter than a polysaccharide. As used herein, oligosaccharides have a degree of polymerisation that is at most about 100, but shorter oligosaccharides are preferred. In preferred embodiments, the oligosaccharide has a degree of polymerisation of 2-75, preferably of 10-60, more preferably of 2-20. Other preferred degrees of polymerisation are 5-18, more preferably 10-15. In highly preferred embodiments the degree of polymerisation is in the range of 2-9, most preferably 4-7. A monodisperse compound has only a single degree of polymerisation (comprising only compounds having that many monomer units), and thus is not a mixture of chains of varying lengths. A polydisperse compound comprises chains of varying lengths. In some preferred embodiments, the oligosaccharide is not monodisperse, or not fully monodisperse. This is particularly attractive when the degree of polymerisation encompasses values in the 4-7 range. It is thought that the polydispersity may assist in achieving good association with the enzyme. In some particular embodiments the degree of polymerisation is 2. For these embodiments, the oligosaccharide is monodisperse. Such embodiments can be attractive to obtain a more precise definition of the oligosaccharide.

Oligosaccharides can be based on hexoses or pentoses or on other saccharides. Preferably the oligosaccharide is an oligohexose, more preferably an oligoketohexose or an oligoaldohexose, even more preferably an oligoketohexose. Examples of hexoses are allose, altrose, glucose, mannose, gulose, idose, galactose, talose, psicose, fructose, sorbose, tagatose, and glucosamine. Examples of ketohexoses are psicose, fructose, sorbose, and tagatose, of which fructose is most preferred. Highly preferred oligosaccharides are primarily linked by β(2→1) bonds, more preferably all non-terminal residues are linked by β(2→1) bonds, most preferably all non-terminal residues and one terminal residue are linked by β(2→1) bonds. In particular embodiments, the terminal residue is linked by a 1,1-glycosidic bond. This is especially preferred when the degree of polymerisation is 2.

It is to be understood that when an oligosaccharide has a terminal residue that differs from the remainder of the oligosaccharide, this terminal residue can sometimes be ignored for naming purposes. For instance, an oligosaccharide having five fructose residues and a terminal glucose residue is generally referred to as an oligofructose, or as an oligoketohexose, or as a fructooligosaccharide, despite the terminal glucose residue being an aldohexose, and not being a fructose.

Examples of oligosaccharides are oligomers of fructose (also known as fructan or inulin), of glucose (also known as glucan or glycogen), of galactose (also known as galactan), or oligomers of dextrin, dextran, mannan, pectin, starch, xanthan gum, isomaltose, or glucosamine (also known as chitosan). Preferred oligosaccharides for use in a storage solution are derived from inulin, isomaltose, or galactan, of which inulin is most preferred. A preferred oligosaccharide derived from isomaltose is isomaltooligosaccharide (IMO), which has a degree of polymerisation in the range of 3-9 with an average near 5. A preferred oligosaccharide derived from galactan is galactooligosaccharide (GOS, also known as oligogalactosyllactose, which is known as a prebiotic), which has a degree of polymerisation in the range of 2-8 with an average near 5. A preferred oligosaccharide derived from inulin is fructooligosaccharide (FOS, also known as oligofructan, which is known as a prebiotic), which has a degree of polymerisation in the range of 2-8 with an average near 5.

A characteristic of FOS is that it comprises terminal glucose residues. FOS is produced by degradation of inulin, a polymer of D-fructose residues linked by β(2→1) bonds with a terminal α(1→2) linked D-glucose. In many natural sources the degree of polymerization of inulin ranges from 10 to 60. Inulin can be degraded enzymatically or chemically to a mixture of oligosaccharides with the general structure Glu-Fruₙ (GFₙ) and Fruₘ (Fₘ), with n ranging from 1 to 7 and with m ranging from 2 to 8. Good results were obtained with FOS, and accordingly in preferred embodiments the oligosaccharide for us in a stabilising method according to the invention comprises terminal aldohexose residues, more preferably terminal glucose residues. Preferably an oligosaccharide comprises an aldohexose residue at one of its termini. Preferably, the terminal aldohexose residue is α(1→2) linked. When the degree of polymerisation is 2, preferably both terminal residues are a terminal aldohexose, even more preferably forming a 1,1-glycosidic bond between two α-glucose units.

Inulin generally has a degree of polymerisation in the range of 2-75, or sometimes 10-60. Fructooligosaccharide (FOS) generally has a degree of polymerisation in the range of 2-20, or sometimes 2-8. Refined inulin (sometimes indicated as CLR by commercial suppliers) generally has a degree of polymerisation in the range of 2-18 or sometimes 5-18. Fractionated refined inulin (sometime indicated as OFP by commercial suppliers) generally has a degree of polymerisation in the range of 2-9. Oligosaccharides with the desired characteristics can be obtained from commercial sources, or can be fractionated or further refined using any known method. Because the range for the degree of polymerisation of FOS encompasses the ranges for refined inulin and for fractionated refined inulin, reference to FOS can be understood as reference to each three of these species, unless context makes it clear that this is not intended. Similarly, refined inulin can refer to both refined inulin as such, and to fractionated refined inulin.

The inventors consider that it is possible that the reductive potential of a terminal aldohexose residue protects the enzyme from oxidative loss of activity. The combination of a terminal aldohexose with oligomers having a predominant degree of polymerisation of about 5 contributes to an optimal interaction between the oligosaccharide and the enzyme, both having matching spatial dimensions. The hydrodynamic radius of a hexameric urease complex is about 14-18 nm (C. Follmer et al., Biophys. Chem., 111 (2004), p 79). Glucose is about 0.8-0.9 nm. Monosaccharides dissociate from the enzyme more because they have fewer interactions. Polysaccharides are dissociated from the enzyme more because their entropic cost for association is higher. The combination of degree of polymerisation and terminal aldohexose contributes to an effective local molarity of the reductive moiety that is high at the enzyme. Particularly when the enzyme is a hydrolase or especially a urease, which has spatial dimensions matching the degree of polymerisation of the oligosaccharide. Accordingly, a preferred oligosaccharide has at least one of the following features:
1) a degree of polymerisation in the range of 2-9;
2) at least 50% of oligosaccharides in the range of 4-8;
3) a modal amount of 5 residues;
4) all non-terminal residues are ketohexose residues
5) at least one terminal residue is an aldohexose residue
6) at least one terminal residue is a ketohexose residue
7) one terminal residue is an aldohexose residue and the other is a ketohexose residue
8) all terminal aldohexose moieties are linked via an α(1→2) bond
9) all ketohexoses are linked to each other by β(2→1) bonds
10) all ketohexose residues are fructose residues
11) all aldohexose residues are glucose residues

The below table provides an overview of preferred embodiments of the oligosaccharide, with reference to the characteristics as described above.

| Oligosaccharide | Characteristics |
|---|---|
| A | 1,4 |
| B | 1, 4, 5, |
| C | 1, 4, 7 |
| D | 2, 4 |
| E | 2, 4, 5 |
| F | 2, 4, 7 |
| G | 3, 4 |
| H | 3, 4, 5 |
| I | 3, 4, 7 |
| J | 1, 2, 3 |
| K | 1, 2, 3, 4 |
| L | 1, 2, 3, 4, 5 |
| M | 1, 2, 3, 4, 7 |
| N | 1, 7 |
| O | 1, 7, 8 |
| P | 2, 7 |
| Q | 2, 7, 8 |
| R | 1, 8, 9 |
| S | 2, 8, 9 |
| T | 1, 2, 3, 8, 9 |
| U | 1, 4, 9 |
| V | 1, 4, 7, 8, 9 |
| W | 2, 4, 7, 8, 9 |
| X | 3, 4, 7, 8, 9 |
| Y | All of 1-9 |
| Z | All of 1-11 |

In preferred embodiments the storage solution comprises 1-80, 2-70, 3-60, 4-50, or 5-40 wt.-% of the oligosaccharide, preferably 10-35 wt.-%, more preferably 15-30 wt.-%. The storage solution can comprise at least 5, 10, preferably 15, more preferably 20, still more preferably 25, more preferably 30, most preferably 35 wt.-% of oligosaccharide. The storage solution can comprise at most 90, 80, 70, 60, 50, preferably 45, more preferably 40, even more preferably 35 wt.-% of oligosaccharide. Particularly good results were obtained in the range of 20-30 wt.-%. Recited percentages are for all comprised oligosaccharides. A skilled person understands that oligosaccharides are inherently mixtures of compounds even when they are of a single type, for instance due to polydispersity or due to their process of formation. Preferably, only a single type of oligosaccharide is comprised, or at least substantially a single type.

The storage solution can comprise various further components. These further components are optional and a skilled person can select components dependent on the intended use of the storage solution. In some embodiments, the storage solution further comprises buffer salts, antioxidants, bacteriostatics, chelators, cryo-protective agents, or serum albumins.

Antioxidants are widely known. Examples of antioxidants are sulfhydryl antioxidants such as glutathione or cysteine; ascorbic acid; propyl gallate; tertiary butylhydroquinone; butylated hydroxyanisole; and butylated hydroxytoluene. Because the invention reduces the need of further antioxidants, in preferred embodiments no antioxidant is added as a further component. When present, it is preferably at 0.1 to 10 wt.-%, 0.5 to 5 wt.-%, or 1 to 2 wt.-%, such as about 1 wt.-%. In preferred embodiments, particularly when the enzyme is immobilized, antioxidant is added as a further component, preferably about 0.1 to 5 wt.-%, preferably a sulfhydryl antioxidant such as glutathione or cysteine. Addition of an antioxidant, preferably a sulfhydryl antioxidant such as glutathione or cysteine, was found to have a positive effect when the degree of polymerisation of the oligosaccharide is 2.

Bacteriostatics are widely known. Examples of bacteriostatics are chloramphenicol, clindamycin, ethambutol, lincosamides, macrolides, nitrofurantoin, novobiocin, oxazolidinone, spectinomycin, sulfonamides, tetracyclines, tigecycline, and trimethoprim. Storage solutions, especially with a pH below 7, particularly below 6 were found to be conveniently microbially stable. Therefore, in preferred embodiments no bacteriostatic is added as a further component. When present, it is preferably at 0.01 to 1 wt.-%, such as about 0.1 wt.-%.

Chelators are widely known. Chelators can inactivate metal ions with regard to detrimental effects they may have on the enzyme to be stabilized. Examples of suitable chelators are dimercaptosuccinic acid (DMSA), 2,3-dimercaptopropanesulfonic acid (DMPS), alpha lipoic acid (ALA), ethylenediaminetetraacetic acid (EDTA), 2,3-dimercaptopropanesulfonic acid (DMPS), and thiamine tetrahydrofurfuryl disulfide (TTFD). Small amounts of chelator, such as 0.01 to 0.1 wt.-%, can stabilize an enzyme, and therefore such a range is preferred.

Cryo-protective agents, also known as cryoprotectants, are widely known. Examples are amino acids, methylamines, polyethylene glycols, polyols, surfactants and monosaccharides or disaccharides. In preferred embodiments no additional cryo-protective agents are used, as the oligosaccharide already acts in this regard.

Serum albumins can serve to stabilise other enzymes present in the stabilizing solution. Examples of serum albumins are bovine serum albumin and human serum albumin. Preferably no serum albumin is present in the storage solution.

The storage solution is preferably buffered, and therefore it can comprise buffer salts. Preferably the storage solution is buffered at a pH in the range of 5.5-8.2, and/or wherein the storage solution is a pharmaceutically acceptable solution. The pH is preferably at least 3, 3.5, 4, 4.5, 5, 5.5, 6, 6.5, 7, or 7.5. The pH is preferably at most 10, 9.5, 9, 8.5, 8, 7.5, or 7. Preferred pH ranges are 4.5-8.5, 5.5-8.5, 5.5-8.0, 5.5-7.5, 5.5-7, 5.5-6.5, and 5.5-6. Most preferably the pH is in the range of 5.5 to 7, with particular preference for the range of 5.5 to 6.5, such as a pH of about 6.

A skilled person knows how to control and adjust the pH of a solution, particularly of a solution that is as elegant as a storage solution. Preferably buffer salts are used. In preferred embodiments, the buffer salts are present in a range of 5 to 500 mM, more preferably from 20 to 350 mM, even more preferably from 50 to 250 mM, still more preferably from 50 to 200 mM, most preferably from 50 to 150 mM. Exemplary storage solutions comprise 100 mM buffer salts, although use of 200 mM is also envisioned. In embodiments the storage solution comprises at least 5, 10, 20, 25, 30, 40, 50, 60, 70, 75, 80, 90, or 100 mM buffer salts. In embodiments the storage solution comprises at most 500, 400, 300, 250, 200, 150, 140, 130, 120, 110, or 100 mM buffer salts.

Suitable buffer salts depend on the desired pH, as is known to a skilled person, who can select a suitable salt. Examples of useful buffer salts are phosphate salts such as sodium phosphate and potassium phosphate (preferably NaH₂PO₄ or KH₂PO₄), citrate, boric acid, ([tris(hydroxymethyl)methylamino]propanesulfonic acid), (tris(hydroxymethyl)aminomethane, (4-(2-hydroxyethyl)-1-piperazineethanesulfonic acid), (3-(N-morpholino)propanesulfonic acid), and (2-(N-morpholino)ethanesulfonic acid). Preferred buffer salts are inorganic buffer salts, such as phosphate salts, more preferably potassium phosphate or sodium phosphate, most preferably potassium phosphate.

In some embodiments, the storage solution is oxygen-free, substantially oxygen-free, or has reduced oxygen content. This can be achieved by sparging with an inert gas such as molecular nitrogen, carbon dioxide, or a noble gas such as argon.

The storage solution can have a dry-matter content of at least 5 wt.-%, based on the total weight of the storage solution, preferably at least 25 wt.-%, or at least 10, 15, 20, 25, 30, 35, 40, 45, 50, 55, 60, 65, 70, 75, 80, 85, or even 90 wt.-%. The storage solution can have a dry-matter content of at most 95 wt.-%, based on the total weight of the storage solution, preferably at most 60 wt.-%, or at most 90, 85, 80, 75, 70, 65, 60, 55, 50, 45, 40, 35, or 30 wt.-%. In some embodiments, the mixture has a dry-matter content of 10-50 wt.-%, based on the total weight of the composition, preferably 10-30 wt. % more preferably 15-25 wt. %. In embodiments, the mixture has a dry-matter content of less than 10 wt. %, based on the total weight of the composition, preferably less than 7 wt. %, less than 5 wt. % or less than 3 wt. %. A preferred method to measure dry matter content is in accordance with ICUMSA GS2/1/3/9-15 (2007).

The storage solution can have any ionic strength, but generally it will be in the range of 1 to 500 mM, or 5 to 400 mM, or 100 to 350 mM. Preferably it is at least 1, 2, 3, 4, 5, 6, 7, 8, 9, or 10 mM, such as at least 10 mM. Preferably it is at most 500, 400, 300, 200, 150, 100, 90, 80, 70, 60, or 50 mM, such as 50 mM.

Any combination of these further components is possible. Preferred is, for example, oligosaccharide with buffer and antioxidant.

Step iii)—Optional Drying

In various preferred embodiments the storage composition is dried. Drying of enzymes can prolong their shelf life as many deterioration processes are known to occur at an enhanced rate in solution. Any known drying method can be used. A drying method can e.g. be spray drying, air drying, coating, foam-drying, desiccation, vacuum drying, vacuum/freeze drying, or freeze-drying, all of which are known to the person skilled in the art. In a preferred embodiment, the drying method is vacuum drying or freeze-drying, of which freeze-drying is most preferred. Freeze drying is also known as lyophilisation.

In one embodiment of a freeze-drying process, the storage composition (solution, suspension, or wetted solid) to be dried preferably is first frozen to an initial freeze-drying temperature in a freezer or on a shelf of a temperature equal to or lower than of −50° C., −40° C., −30° C., −20° C. or −10° C. A preferred initial shelf temperature is equal to or lower than of −50° C. or −40° C. The storage composition to be dried may be subjected to fast freezing by immediately placing (a container/vial comprising) the solution on the shelf having an initial shelf temperature as indicated above. Alternatively, the storage composition to be dried may be subjected to slow freezing by placing (a container/vial comprising) the storage composition on the shelf having a temperature above 0° C., e.g. 2, 4 or 6° C., and then slowly freezing the storage composition to the initial freeze-drying temperature as indicated above, by reducing the temperature, preferably at a rate of about 0.5, 1 or 2° C. per minutes. The storage composition to be dried may be brought to a pressure of 100 microbar or lower. When the set pressure has been reached, the shelf temperature may be increased to higher temperatures. The shelf temperature may e.g. be increased at a rate of e.g. 0.05, 0.1 or 0.2° C. per minute to a temperature of 5, 10 or 15 or ºC above the initial freeze-drying temperature. The primary drying step is preferably ended when no pressure rise is measured in the chamber. Preferably at that moment, the shelf temperature may be increased to e.g. 5, 10, 15, 20 or 25° C. at a rate of e.g. 0.01, 0.02 or 0.05° C. per minute and optionally in one or more steps. During the secondary drying phase the temperature is preferably kept at this value until no pressure rise can be detected.

In one embodiment of a vacuum-drying process, the storage composition (solution, suspension, or wetted solid) to be dried preferably is at a temperature in the range of about 5-25° C., e.g. room temperature or more preferably at a temperature in the range of about 10-20° C., e.g. a temperature of about 15° C. The pressure is then reduced, e.g. to a pressure of less than 1, 0.5, 0.2, 0.1, 0.05 mbar. Once under reduced pressure the temperature of the storage composition being dried can be decreased to a temperature that can be below 0° C. but that is (just) above the eutectic temperature of the storage composition to prevent freezing. When no pressure rise is measured in the chamber, the temperature of the storage composition can be increased to e.g. 5, 10, 15, 20 or 25° C. at a rate of e.g. 0.01, 0.02 or 0.05° C. per minute and optionally in one or more steps. The temperature is preferably kept at this value till no pressure rise can be detected.

Dry matter can be described by its water activity. Preferably, a dried composition according to the invention has a water activity less than 1, more preferably less than 0.9, even more preferably less than 0.8, still more preferably less than 0.7, even more preferably less than 0.6, more preferably less than 0.5, still more preferably less than 0.4, most preferably less than 0.3. Water activity can be determined using any method known in the art, such as using a hygrometer. Dry matter can also be described by its weight loss on drying (LOD). Matter with low LOD can be considered dry. A preferred method to measure dry matter content is in accordance with ICUMSA GS2/1/3/9-15 (2007).

A dried composition can be a fluid powder, a viscous powder, or a paste. The dried composition can be further processed, for instance to bring it in conformance with regulatory requirements. A preferred dried composition is bio-safe. In some embodiments the storage composition is not dried. In some embodiments the storage composition is dried.

Step iv)—Optional Storage of the Storage Composition

In preferred embodiments, the storage composition is stored after it has been obtained. It can be advantageously stored for many days with only a low loss of activity for the stored enzyme. In some embodiments, the storage composition is stored for at least two days. In other embodiments the storage composition is stored for at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, or 120 days. Preferably, storage can be for at least two years. In some embodiments the storage composition is stored for at most 3 years, preferably at most 2 years, preferably at most 365 days, in other embodiments the storage composition is stored for at most 350, 300, 250, 200, 150, 125, 120, 115, 110, 105, or 100 days.

An advantage of the invention is that storage of the storage composition can be under standard conditions. Storage can be at 20° C., or at at least 0, 5, 10, 15 or 20° C., preferably at least 15° C. Storage is preferably at at most 50° C., or at most 40° C., or at most 30° C., more preferably at most 25° C. Storage is preferably at standard atmospheric pressure.

The invention improves the stability of an enzyme, particular during storage. This is apparent from the maintained enzyme activity that can be observed when enzymes have been stored after having been contacted with a storage solution. In this context, stabilisation preferably refers to retained enzymatic activity. Stability is said to have been improved when activity after storage is retained to a greater extent as compared to an enzyme that has been stored under identical conditions except without having been contacted with a storage solution.

Enzymatic activity can be assayed using any method that a skilled person knows to be suitable for the selected enzyme. For assessing retained activity, it is preferred that an enzyme sample is stored separate from another enzyme sample, where one enzyme sample is stored according to a stabilizing method according to the invention, and the other is stored under identical conditions except without having been contacted with a storage solution. A preferred method for assaying enzymatic activity is via spectroscopy when using a chromogenic substrate, for instance such as described in the examples.

In preferred embodiments, the stored enzyme retains at least 10% of its original activity after a reference period. More preferably the enzyme retains at least 15% of its original activity, or 20, 25, 30, 35, 40, 45, 50, 55, 60, 65, 70, 75, 80, 85, 90, 91, 92, 93, 94, 95, 96, 97, 98, 99, or 100% of its original activity. Preferably at least 75% of original activity is retained. A skilled person understands that some loss of activity is acceptable, and that there is benefit in reducing the loss of activity. The reference period is preferably at least 2 days, or it is 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, or 120 days. For a good indication, the reference period can be 25 days.

In other embodiments the reference activity is the activity after the stabilisation method according to the invention has been performed, assaying the initial activity at the same day (T=0). Here, loss of activity is preferably at most 60%, more preferably at most 50%, even more preferably at most 40%, or even 35, 30, 25, 20, 15, 10, 5, 4, 3, 2, or 1%. In preferred embodiments the storage composition is stored for at least 25 days, wherein the enzyme retains at least 75% of its original activity after the storage. In preferred embodiments the storage composition is stored for at least 40 days, wherein the enzyme retains at least 50% of its original activity after the storage. In preferred embodiments the storage composition is stored for at least 60 days, wherein the enzyme retains at least 50% of its original activity after the storage.

The invention also improves the stability of an enzyme during sterilization. Sterilization is a procedure that removes, deactivates, or kills all life, particularly microbial life, in a sample, to render it aseptic. In view of this goal, sterilization conditions are generally harsh and often promote material degradation. Sterilization can be achieved through various means, including heat, chemicals, irradiation, high pressure, and filtration. In the context of the present invention, use of sterilizing chemicals is not compatible with the intended (medical) use of the enzyme. Similarly, filtration is problematic, for instance in view of the size of the enzyme, particularly for immobilized enzyme. A particularly useful means of sterilization is irradiation with ionizing radiation. Preferred methods of irradiation sterilization are gamma irradiation, and electron beam sterilization. Gamma irradiation is most preferred.

Single-use systems used in medical applications are generally required to be sterilized before use. Therefore, a preferred composition according to the invention is a sterilized composition. Also, the optional storage step is preferably initiated by a sterilization step, preferably an irradiation sterilization step.

Gamma rays are a form of electromagnetic radiation, and use of gamma rays in sterilization is known in the art (see for instance WO2020097711). The primary industrial sources of gamma rays are radionuclide elements such as ⁶⁰Co. Gamma radiation passes readily through materials and kills bacteria by breaking the covalent bonds of bacterial DNA, typically involving radical oxidative processes. The absorbed radiation dose is measured in kiloGrays (kGy).

Electron beam irradiation is a form of ionizing radiation that is characterized by its low penetration and high-dosage rates. Electron beam sterilization is known in the art (see for instance WO2020241758). The beam is a concentrated, highly charged stream of electrons and is generated by accelerators capable of producing continuous or pulsed beams. As the material being sterilized passes the beam, energy from the electrons is absorbed, altering various chemical bonds, damaging DNA, and destroying the reproductive capabilities of microorganisms.

With irradiation sterilization being damaging to biomaterial, the inventors were surprised to find that enzymatic activity was preserved to a much greater extent than expected when the methods and compositions according to the invention were used with for instance gamma sterilization.

### Particular Embodiments

The invention provides a method for storing an enzyme, the method comprising the steps of:
I) providing a composition as defined elsewhere herein, or a cartridge as described elsewhere herein, and
II) storing the composition or cartridge for at least 2 days. Preferably it is stored for at least 7 days, even more preferably for at least 15 days. Features and definitions are as provided elsewhere herein.

In preferred embodiments, the stabilizing method comprises steps i) and ii).

In preferred embodiments, the stabilizing method comprises steps i), ii), and iv).

In preferred embodiments, the stabilizing method comprises steps i), ii), and iii).

In preferred embodiments, the stabilizing method comprises steps i), ii), iii), and iv).

In preferred embodiments the storage solution comprises or consists of water and about 25 wt.-% of the oligosaccharide; and optionally buffer salts, preferably phosphate buffer salts.

In preferred embodiments the storage solution comprises or consists of water and about 20 to 35 wt.-% of the oligosaccharide; and optionally buffer salts, preferably phosphate buffer salts; and optionally antioxidants.

In preferred embodiments the storage solution comprises or consists of water and about 20 to 30 wt.-% of the oligosaccharide; and optionally buffer salts, preferably phosphate buffer salts.

In preferred embodiments the storage solution comprises or consists of water and about 20 to 30 wt.-% of the oligosaccharide; and buffer salts, preferably phosphate buffer salts.

In preferred embodiments the storage solution comprises or consists of water and about 25 wt.-% of the oligosaccharide; and 50-250 mM buffer salts, preferably phosphate buffer salts; and optionally antioxidants.

In preferred embodiments the storage solution comprises or consists of water and about 20 to 35 wt.-% of the oligosaccharide; and 50-200 mM buffer salts, preferably phosphate buffer salts.

In preferred embodiments the storage solution comprises or consists of water and about 20 to 30 wt.-% of the oligosaccharide; and 75-200 mM buffer salts, preferably phosphate buffer salts.

In preferred embodiments the storage solution comprises or consists of water and about 20 to 30 wt.-% of the oligosaccharide; and 75-150 mM buffer salts, preferably phosphate buffer salts.

In preferred embodiments the storage solution comprises or consists of water and about 20 to 30 wt.-% of the oligosaccharide; and 75-125 mM buffer salts, preferably phosphate buffer salts.

In preferred embodiments the storage solution comprises or consists of water and about 20 to 30 wt.-% of the oligosaccharide; and about 100 mM buffer salts, preferably phosphate buffer salts; and optionally antioxidants.

In preferred embodiments the storage solution comprises or consists of water and about 25 wt.-% FOS; and optionally buffer salts, preferably phosphate buffer salts.

In preferred embodiments the storage solution comprises or consists of water and about 20 to 35 wt.-% FOS; and optionally buffer salts, preferably phosphate buffer salts; and optionally antioxidants.

In preferred embodiments the storage solution comprises or consists of water and about 20 to 30 wt.-% FOS; and optionally buffer salts, preferably phosphate buffer salts.

In preferred embodiments the storage solution comprises or consists of water and about 20 to 30 wt.-% FOS; and buffer salts, preferably phosphate buffer salts.

In preferred embodiments the storage solution comprises or consists of water and about 25 wt.-% FOS; and 50-250 mM buffer salts, preferably phosphate buffer salts; and optionally antioxidants.

In preferred embodiments the storage solution comprises or consists of water and about 20 to 35 wt.-% FOS; and 50-200 mM buffer salts, preferably phosphate buffer salts.

In preferred embodiments the storage solution comprises or consists of water and about 20 to 30 wt.-% FOS; and 75-200 mM buffer salts, preferably phosphate buffer salts.

In preferred embodiments the storage solution comprises or consists of water and about 20 to 30 wt.-% FOS; and 75-150 mM buffer salts, preferably phosphate buffer salts.

In preferred embodiments the storage solution comprises or consists of water and about 20 to 30 wt.-% FOS; and 75-125 mM buffer salts, preferably phosphate buffer salts.

In preferred embodiments the storage solution comprises or consists of water and about 20 to 30 wt.-% FOS; and about 100 mM buffer salts, preferably phosphate buffer salts.

In preferred embodiments the storage solution comprises or consists of water and about 25 wt.-% FOS or fractionated refined inulin, preferably fractionated refined inulin; and optionally buffer salts, preferably phosphate buffer salts; and optionally antioxidants.

In preferred embodiments the storage solution comprises or consists of water and about 20 to 35 wt.-% FOS or fractionated refined inulin, preferably fractionated refined inulin; and optionally buffer salts, preferably phosphate buffer salts.

In preferred embodiments the storage solution comprises or consists of water and about 20 to 30 wt.-% FOS or fractionated refined inulin, preferably fractionated refined inulin; and optionally buffer salts, preferably phosphate buffer salts.

In preferred embodiments the storage solution comprises or consists of water and about 20 to 30 wt.-% FOS or fractionated refined inulin, preferably fractionated refined inulin; and optionally buffer salts, preferably phosphate buffer salts.

In preferred embodiments the storage solution comprises or consists of water and about 25 wt.-% FOS or fractionated refined inulin, preferably fractionated refined inulin; and 50-250 mM buffer salts, preferably phosphate buffer salts; and optionally antioxidants.

In preferred embodiments the storage solution comprises or consists of water and about 20 to 35 wt.-% FOS or fractionated refined inulin, preferably fractionated refined inulin; and 50-200 mM buffer salts, preferably phosphate buffer salts.

In preferred embodiments the storage solution comprises or consists of water and about 20 to 30 wt.-% FOS or fractionated refined inulin, preferably fractionated refined inulin; and 75-200 mM buffer salts, preferably phosphate buffer salts.

In preferred embodiments the storage solution comprises or consists of water and about 20 to 30 wt.-% FOS or fractionated refined inulin, preferably fractionated refined inulin; and 75-150 mM buffer salts, preferably phosphate buffer salts.

In preferred embodiments the storage solution comprises or consists of water and about 20 to 30 wt.-% FOS or fractionated refined inulin, preferably fractionated refined inulin; and 75-125 mM buffer salts, preferably phosphate buffer salts.

In preferred embodiments the storage solution comprises or consists of water and about 20 to 30 wt.-% FOS or fractionated refined inulin, preferably fractionated refined inulin; and about 100 mM buffer salts, preferably phosphate buffer salts.

In preferred embodiments the storage solution comprises or consists of water and about 20 to 30 wt.-% FOS or fractionated refined inulin, preferably fractionated refined inulin; and about 100 mM buffer salts, preferably phosphate buffer salts; and antioxidants.

In preferred embodiments the storage solution comprises or consists of water and about 20 to 30 wt.-% FOS or fractionated refined inulin, preferably fractionated refined inulin; and about 100 mM buffer salts, preferably phosphate buffer salts; and no additional antioxidants.

In preferred embodiments the storage solution comprises or consists of water and about 23 to 30 wt.-% trehalose; and optionally buffer salts, preferably phosphate buffer salts.

In preferred embodiments the storage solution comprises or consists of water and about 23 to 30 wt.-% oligosaccharide having a degree of polymerisation of 2; and optionally buffer salts, preferably phosphate buffer salts; and 1-3 wt.-% antioxidants, preferably sulfhydryl antioxidants, more preferably glutathione.

In preferred embodiments the storage solution comprises or consists of water and about 23 to 30 wt.-% oligosaccharide having a degree of polymerisation of 2 and comprising a terminal glucose residue; and optionally buffer salts, preferably phosphate buffer salts; and 1-3 wt.-% antioxidants, preferably sulfhydryl antioxidants, more preferably glutathione.

In preferred embodiments the storage solution comprises or consists of water and about 23 to 30 wt.-% trehalose; and optionally buffer salts, preferably phosphate buffer salts; and 1-3 wt.-% antioxidants, preferably sulfhydryl antioxidants, more preferably glutathione.

In preferred embodiments the storage solution comprises or consists of water and about 23 to 30 wt.-% trehalose; and optionally buffer salts, preferably phosphate buffer salts; and 1-2 wt.-% antioxidants, preferably sulfhydryl antioxidants, more preferably glutathione.

In preferred embodiments the storage solution comprises or consists of water and about 23 to 30 wt.-% trehalose; and 50-250 mM buffer salts, preferably phosphate buffer salts; and 1-2 wt.-% antioxidants, preferably sulfhydryl antioxidants, more preferably glutathione.

In preferred embodiments the storage solution comprises or consists of water and about 25 to 30 wt.-% trehalose; and optionally buffer salts, preferably phosphate buffer salts.

In preferred embodiments the storage solution comprises or consists of water and about 25 to 30 wt.-% oligosaccharide having a degree of polymerisation of 2; and optionally buffer salts, preferably phosphate buffer salts; and 1-3 wt.-% antioxidants, preferably sulfhydryl antioxidants, more preferably glutathione.

In preferred embodiments the storage solution comprises or consists of water and about 25 to 30 wt.-% oligosaccharide having a degree of polymerisation of 2 and comprising a terminal glucose residue; and optionally buffer salts, preferably phosphate buffer salts; and 1-3 wt.-% antioxidants, preferably sulfhydryl antioxidants, more preferably glutathione.

In preferred embodiments the storage solution comprises or consists of water and about 25 to 30 wt.-% trehalose; and optionally buffer salts, preferably phosphate buffer salts; and 1-3 wt.-% antioxidants, preferably sulfhydryl antioxidants, more preferably glutathione.

In preferred embodiments the storage solution comprises or consists of water and about 25 to 30 wt.-% trehalose; and optionally buffer salts, preferably phosphate buffer salts; and 1-2 wt.-% antioxidants, preferably sulfhydryl antioxidants, more preferably glutathione.

In preferred embodiments the storage solution comprises or consists of water and about 25 to 30 wt.-% trehalose; and 50-250 mM buffer salts, preferably phosphate buffer salts; and 1-2 wt.-% antioxidants, preferably sulfhydryl antioxidants, more preferably glutathione.

### Composition and Other Products

The invention provides a composition comprising an enzyme as defined above and an oligosaccharide as defined above. Such a composition is referred to hereinafter as a composition according to the invention. Such a composition is preferably a pharmaceutical composition. Such a composition is preferably a dried composition.

The methods of the invention for producing a dried formulation of an enzyme are preferably aimed at minimizing the loss of activity of the enzyme upon drying of the formulation. Preferably the methods of the invention as well as the compositions themselves are also aimed at minimizing the loss of activity of the enzyme upon subsequent storage of the composition obtainable with the methods of the invention. The methods of the invention are thus preferably methods for producing compositions according to the invention, which are stable formulations of enzymes, i.e. formulations with a long or extended shelf-life, preferably under refrigerated conditions (e.g. 2-10° C.), at room temperature (e.g. 18-25° C.), or even at elevated temperatures (e.g. 32-45° C.) as may occur in tropical regions. Room temperature is preferred.

Compositions and pharmaceutical compositions according to the invention may be manufactured by processes well known in the art; e.g., by means of conventional mixing, dissolving, granulating, dragee-making, levigating, emulsifying, encapsulating, entrapping or lyophilizing processes, which may result in liposomal formulations, coacervates, oil-in-water emulsions, nanoparticulate/microparticulate powders, or any other shape or form. Compositions for use in accordance with the invention thus may be formulated in conventional manner using one or more physiologically acceptable carriers comprising excipients and auxiliaries that facilitate processing of the active compounds into preparations which can be used pharmaceutically.

Alternatively, one or more components of the composition may be in powder form for constitution with a suitable vehicle, e.g., sterile pyrogen-free water, before use. Components of the composition may be supplied separately.

The compositions or pharmaceutical compositions according to the invention also may comprise suitable solid or gel phase carriers or excipients. Examples of such carriers or excipients include but are not limited to calcium carbonate, various sugars, starches, cellulose derivatives, gelatin, and polymers such as polyethylene glycols. A sorbent such as a urea sorbent can be envisaged as a carrier.

A pharmaceutical composition according to the invention can also comprise a further pharmaceutically active substance, preferably a further pharmaceutically active substance for the treatment of a disease or condition associated with accumulation of urea or with improper clearance of urea, such as acute kidney failure or end stage kidney disease (ESKD).

The composition according to the invention can comprise further components. In preferred embodiments of the composition the enzyme is immobilized. In preferred embodiments of the composition the enzyme is urease. Additional examples of further components are ion exchangers such as zirconium salts (for instance zirconium phosphate), and antioxidants such as inorganic antioxidants, which are preferably inorganic materials that can scavenge molecular oxygen from an atmosphere, or organic antioxidants such as glutathione, cysteine or ascorbic acid.

In preferred embodiments of the composition, the enzyme is an amidohydrolase, preferably a urease, or the oligosaccharide is an oligohexose, preferably an oligoketohexose or an oligoaldohexose, more preferably an oligoketohexose. More preferably the enzyme is an amidohydrolase, preferably a urease, and the oligosaccharide is an oligohexose, preferably an oligoketohexose or an oligoaldohexose, more preferably an oligoketohexose, most preferably FOS.

In preferred embodiments the composition has a dry-matter content of at least about 99%, or is a dry composition that can be a powder. In some embodiments, the composition may be in the form of a solution, a suspension, an emulsion, a powder, or a paste. As used herein, paste refers to a semi-liquid of high viscosity, which may be a colloidal suspension, emulsion, and/or a dispersion of aggregated material. It will be understood by the skilled person that the dry-matter content range wherein the composition has the form of a paste depends inter alia on the amount and type of other components present in the composition. It is within the capabilities of the skilled person to adjust the dry-matter content to obtain a paste. In embodiments, the composition is in the form of a paste, wherein the paste has a dry-matter content of 10-50 wt. %, based on the total weight of the composition, preferably 10-30 wt. % or 15-25 wt. %.

In preferred embodiments, the composition is packaged, preferably aseptically packaged. Preferably the packaging includes instructions on use of the composition in dialysis applications.

A composition according to the invention can advantageously be used in renal replacement therapy, such as peritoneal dialysis or hemodialysis. During such use, the composition is generally present in a cartridge or membrane, which can be replaceably inserted in a (hemo)dialysis device. Accordingly, the invention provides a cartridge for use in a dialysis device, comprising a composition according to the invention. Such a dialysis device can be a hemodialysis device or a device for regeneration of peritoneal dialysate in peritoneal dialysis. Accordingly, the invention provides a membrane for use in a dialysis device, comprising a composition according to the invention. Such a dialysis device can be a hemodialysis device or a device for regeneration of peritoneal dialysate in peritoneal dialysis. Accordingly, the invention provides a dialysis device comprising a composition according to the invention, or a cartridge according to the invention. Such a dialysis device can be a hemodialysis device or a device for regeneration of peritoneal dialysate in peritoneal dialysis.

Besides the composition according to the invention (as comprised in the cartridge, the membrane, or the dialysis device) such cartridges, membranes, and dialysis devices are known in the art. In particular embodiments, the cartridge is a disposable cartridge. In particular embodiments, the cartridge is a regenerable cartridge. Cartridges can also be referred to as cassettes. The cartridge is preferably adaptable to be used with various different types of components and to be arranged in a variety of ways. A cartridge may comprise further components such as sorbents such as urea sorbents. By removing urea as a waste solute, the cartridge at least partially regenerates the dialysate and/or filtrate used during dialysis. The cartridge preferably includes a body having a fluid inlet and a fluid outlet. The interior of the cartridge is preferably constructed and arranged so that fluid entering the interior from the inlet flows through the composition and subsequently through the outlet. Herein the composition preferably comprises immobilized enzymes such as immobilized urease.

A dialysis device is a closed, sterile system. It comprises one or two fluid circuits. It usually comprises two circuits: a so-called patient loop, which is a fluid circuit that is arranged for a subject's fluid such as blood or peritoneal dialysate to flow through it, and a so-called regeneration loop, wherein a dialysis fluid such as dialysate and/or filtrate is circulated through a cartridge as described above. The two circuits are separated from each other by a (semi-permeable) membrane, through which waste solutes can diffuse or pass from the subject's fluid into the dialysis fluid. Air, moisture, pathogens, and fluids from the environment around the dialysis device cannot enter into the fluid circuits. The dialysis system only permits fluids (such as ultrafiltrate) and air to exit or enter these fluid circuits under controlled circumstances, preferably under strictly controlled circumstances.

### Medical Use

Many enzymes can be used in known medical treatments. The compositions of the invention provide more stable alternatives for use in such treatments. Thus the invention provides the medical use of compositions according to the invention. This use is preferably for use in the treatment of a disease or condition associated with accumulation of urea or with improper clearance of urea. Such a composition is referred to herein as a product for use according to the invention.

In particular embodiments of this aspect, the invention provides a composition according to the invention, for use as a medicament for use in the treatment of a disease or condition associated with accumulation of urea or with improper clearance of urea. In further particular embodiments of this aspect, the invention provides a composition according to the invention, for use as a medicament, wherein the composition is for removing urea from a subject.

Treatment of a disease or condition can be the amelioration, suppression, prevention, delay, cure, or prevention of a disease or condition or of symptoms thereof, preferably it shall be the suppression of symptoms of a disease or condition. Urea can accumulate or can be insufficiently cleared in case of kidney failure. Examples of diseases or conditions associated with accumulation of urea or with improper clearance of urea are end stage kidney disease (ESKD); severe acute kidney failure; severe acute kidney injury (AKI); increased hepatic production of urea for example due to gastro-intestinal haemorrhage; increased protein catabolismfor example due to trauma such as major surgery or extreme starvation with muscle breakdown; increased renal reabsorption of urea, for example due to any cause of reduced renal perfusion, for example congestive cardiac failure, shock, severe diarrhea; iatrogenic conditions due to urea infusion for its diuretic action, due to drug therapy leading to an increased urea production such as treatment with tetracyclines or corticosteroid; chronic kidney failure; and urinary outflow obstruction.

Products for use according to the invention can be administered to a subject in need thereof, allowing the product for use according to the invention to bind nucleophilic waste solutes in the subject. Such administration is preferably administration of an effective amount. The use of for instance sorbents in such a method is known in the art (Gardner et al., Appl Biochem Biotechnol. 1984; 10:27-40.)

Administration can be via methods known in the art, preferably via oral ingestion in any formulation known in the art such as a capsule, pill, lozenge, gel capsule, push-fit capsule, controlled release formulation, or via rectal administration as a clyster or suppository. It can be once per week, 6, 5, 4, 3, 2, 1 time per week, daily, twice daily, or three times per day, or four times per day.

Products for use according to the invention are suitable for use in a method of treatment. Such a method of treatment can be a method comprising the step of administering to a subject, preferably a subject in need thereof, an amount, preferably an effective amount, of product for use according to the invention.

With respect to dialysis therapy, the present invention can be used in a variety of different dialysis therapies to treat kidney failure. Dialysis therapy as the term or like terms are used throughout the text is meant to include and encompass any and all forms of therapies to remove waste, toxins and excess water from the subject suffering from a disease or condition. It also provides homeostasis. The hemo therapies, such as hemodialysis, hemofiltration and hemodiafiltration, include both intermittent therapies and continuous therapies used for continuous renal replacement therapy (CRRT). The continuous therapies include, for example, slow continuous ultrafiltration (SCUF), continuous venovenous hemofiltration (CVVH), continuous venovenous hemodialysis (CVVHD), continuous venovenous hemodiafiltration (CVVHDF), continuous arteriovenous hemofiltration (CAVH), continuous arteriovenous hemodialysis (CAVHD), continuous arteriovenous hemodiafiltration (CAVHDF), continuous ultrafiltration periodic intermittent hemodialysis or the like. The present invention can also be used during peritoneal dialysis including, for example, continuous ambulatory peritoneal dialysis (CAPD), automated peritoneal dialysis (APD), continuous flow peritoneal dialysis and the like. Further, although the present invention, in an embodiment, can be utilized in methods providing a dialysis therapy for subjects having acute or chronic kidney failure or disease, it should be appreciated that the present invention can also be used for acute dialysis needs, for example, in an emergency room setting. Cartridges as described herein are preferred for such applications. However, it should be appreciated that the compositions of the present invention can be effectively utilized with a variety of different applications, physiologic and non-physiologic, in addition to dialysis.

### General Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value more or less 10% of the value, optionally more or less 1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37º C (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use are suitable for use in methods of treatment.

Throughout this application, (hemo)dialysis refers to both hemodialysis and dialysis. In general, a dialysis device can refer to any type of dialysis device as described herein.

The present invention has been described above with reference to a number of exemplary embodiments. Modifications, combinations, and alternative implementations of some parts or elements are possible, and are included in the scope of protection as defined in the claims. All citations of literature and patent documents are hereby incorporated by reference.

### DESCRIPTION OF DRAWINGS

FIG. 1—urease activity monitored over prolonged storage in the presence of different concentrations of glucose. Urease was suspended in the indicated buffer at pH 6-7, filtered, and dried, after which it was stored at 20° C. and assayed at indicated time points. Enzyme activity halved after only a few days for all conditions tested.

FIG. 2—urease activity in the presence of 5% glutathione with or without 25% glucose. Even with both additives, activity fell by about 60% after 30 days.

FIG. 3—urease activity over time, in the presence of oligosaccharide (here oligofructose, fractionated refined inulin with a degree of polymerisation in the range of 2-9) as compared to glucose. The oligosaccharide stabilises at about 2 U/mg and remains there for a prolonged time, while glucose falls below 1 U/mg within 20 days.

FIG. 4—urease activity after storage, after being dried from different carbohydrates at 25 wt.-%. Storage was at 20° C. The oligosaccharide outperforms the monosaccharides and disaccharides.

FIG. 5—urease activity after 9 or 10 days of storage at 20° C. The urease was stored at varying weight percentages of oligosaccharide (same as for FIG. 3). Stability is negatively affected when more than 40% or less than 20% is used.

FIG. 6—the addition of an additional reducing agent does not increase the stability of urease when oligosaccharides are used instead of glucose.

FIG. 7—different compositions of 25 wt.-% stabilising agent were used for suspending immobilized urease in 100 mM phosphate buffer at pH 6 (sodium phosphate for disaccharides, potassium phosphate for fructooligosaccharide). The urease was filtered and dried afterwards, and tested for activity in time. Trehalose provided more stability than lactose. Fructooligosaccharide was most effective.

FIG. 8—different compositions of 25 wt.-% or 26 wt.-% stabilising agent were used prior to drying immobilized urease as described for other experiments. Fructooligosaccharide provided long lasting stabilisation. Trehalose provided less stabilisation, which could be improved when GSH (5 wt.-%) was also present—although not to the level of fructooligosaccharide.

### EXAMPLES

### Example 1—Experimental Methods

Provision of urease—urease can be procured from commercial sources, or it can be isolated from organisms such as Jack Bean (Canavalia ensiformis) using known methods. Urease can be used as a free enzyme, or can be immobilised using known methods, for instance those of WO2011102807 or U.S. Pat. No. 8,561,811 or WO2016126596 or Zhang et al., DOI: 10.1021/acsomega.8b03287 or v. Gelder et al, 2020, Biomaterials 234, 119735.

Provision of oligosaccharides—oligosaccharides are commercially available, or can be isolated from organisms such as chicory, using known methods. Here, isolation of inulin from chicory root was achieved by first, extraction using deionized water at elevated temperature, followed by carbonation (0.1 M Ca(OH)₂ and CO₂ gas). This was filtered to remove some small molecular weight components and washed on a subsequent anion and cation exchange bed to further exclude other components such as tannins and pigments. Partial hydrolysis of inulin into FOS with different distributions in degree of polymerisation were achieved by exposing the inulin to acidic conditions (pH 2-3) at elevated temperatures (70-90° C.) for 30-90 minutes. Derived mixtures were fractionated using gel-filtration chromatography (p.e. using a Biogel P2 or Sephadex G50 column). Refined inulin has about 5 to 18 monomers, with the majority of oligomers in the 10-15 range. Fractionated refined inulin has about 2-9 monomers, with the majority of oligomers in the 4-7 range.

Urease activity—the activity of urease can be determined by quantification of the amount of ammonia formed in time when urease is placed in a aqueous 100 mM potassium phosphate buffer at pH 7.5 in the presence of 15 mM urea at room temperature (20° C.). Samples are taken from this mixture and pipetted into a 96-wells plate. To the sample a 1:1 (v/v) cooled (0° C.) and fresh mixture of reagent A and reagent B is added, which allows ammonia to undergo the Berthelot reaction, yielding a green dye. Absorbance of the solution at 620 nm is used to quantify the ammonia concentration, which correlates with urease activity.

Reagent A: sodium salicylate (4.80 g, 30 mmol), sodium nitroprusside dihydrate (0.54 g, 1.8 mmol), EDTA (0.373 g, 1.28 mmol) in 500 mL de-ionized water.

Reagent B: sodium hydroxide (3.0 g, 75 mmol) and sodium hypochlorite 5-15% (10.2 g, 8.4 mL) in 500 mL de-ionized water.

Buffered 15 mM urea solution: dibasic potassium phosphate (7.26 g, 41.7 mmol), monobasic potassium phosphate (1.13 g, 8.3 mmol) and urea (0.45 g, 7.5 mmol) in 500 mL de-ionized water.

Procedure I: urease is dissolved (or suspended for immobilized enzyme) in de-ionized water (10 mg/mL). Of this solution 40 μL (0.4 mg urease) is pipetted in a 50 mL disposable tube. The buffered 15 mM urea solution (10 mL) was added to the tube (at t=0) and the samples were placed on a shaker at 200 rpm. At several time points (4, 8, 12 and 16 minutes) ammonia concentration of the solution in the tubes was determined by pipetting 5 L of the solution in a 96-well plate. To each well 300 μL of a 1:1 (v/v) mixture of reagent A and reagent B (mixture is kept on ice) was added and the mixture was incubated for 20-40 minutes at RT after which the absorption was measured at 620 nm. The ammonia concentration in the tube was plotted against time and the slope of the four time points is calculated with linear regression. The specific activity of the urease was determined with the following formula: Activity=(volume*slope)/((1−LOD)*weight) In which: Activity is the enzymatic activity of urease in U/mg. Volume is the amount of buffered 15 mM urea solution, typically 10 mL. Slope is the slope in the plot of ammonia concentration (mM) versus time (minutes). LOD is the weight loss on drying. Typically 0.55 (55%) for immobilized urease and 0 for urease is used. Weight is the amount of (immobilized) urease in mg in the tube.

Procedure II: determination of the activity of immobilized urease. A 50 mL disposable tube was charged with 30-40 mg of dried immobilized urease (see procedure V). A buffered 15 mM urea solution was spiked with ammonium chloride to a concentration of 3.5-4.0 mM (100 mg per 500 mL), and 10 mL of this solution is added to the tube (at t=0 min). The procedure is continued as described in procedure I.

Shelf life assay—Procedure III: Shelf life of urease samples; Jack Bean Urease (Sigma Aldrich, ˜8 U/mg) was weighed in 5-10 different 1.5 mL disposable tubes and the weight was noted (5-10 mg) for each tube. The samples were closed under air and placed in the dark cabinet at 20° C. At the indicated time points, one tube was removed and the urease present in the tube was dissolved in de-ionized water to make a 10 mg/mL solution, of which the activity was measured in duplo according to procedure I.

Procedure IV: Shelf life of lyophilized urease and urease:oligosaccharide mixtures. In a 50 mL disposable tube 150 mg Jack Bean urease (Sigma Aldrich, ˜8 U/mg) was placed and dissolved in 5 mL de-ionized water. Similarly, in a tube 150 mg urease was placed and 300 mg fractionated refined inulin with a degree of polymerisation in the range of 2-9 was added and the mixture was dissolved in 5 mL de-ionized water. The contents of both tubes were lyophilized overnight. The dry content of both tubes were distributed over 1.5 mL disposable tubes and the shelf life of the samples was monitored similarly as described in procedure III. The activity of the samples was determined with procedure I.

Procedure V: Shelf life of immobilized urease with various stabilizers, A stabilization solution is prepared by mixing a buffer and additives to make a total of 20 grams (see table 1.1). Immobilized urease (prepared as described in U.S. Pat. No. 8,561,811, 1.0 g) was suspended in the stabilization solution (20 g) at 20° C. and placed on a shaker at 200 rpm. After 15 minutes the suspension was vacuum filtrated over filter paper (Whattman), resulting in a white residue of wet immobilized urease, which typically had a water content of ˜55%. To reduce the water content to about 10-15%, a portion of the wet residue (500 mg) was placed in a 50 mL disposable tube and dried. The final mixture (having the reduced water content) was divided over 5-10 separate 1.5 mL disposable tubes, closed under air and stored in the dark at 20° ° C. At time intervals a tube was removed from the storage and the activity of the material stored in that tube was determined in duplo following procedure II.

For each batch of samples the contents of the stabilization solution and storage conditions are specified in table 1.1.

TABLE 1.1
solutions and compositions used

| | Buffer | | |
|---|---|---|---|
| Entry | (K or Na phosphate) | Additive* | Solution |
| 1 | — | — | 20 mL de-ionized water |
| 2 | — | 25% Glucose | 15 mL DIW, 5 gram glucose |
| 3 | — | 10% Glucose | 18 mL DIW, 2 gram glucose |
| 4 | — | 40% Glucose | 12 mL DIW, 8 gram glucose |
| 5 | 100 mM K, pH 9.0 | — | 20 mL buffer |
| 6 | 100 mM K, pH 7.9 | — | 20 mL buffer |
| 7 | 100 mM K, pH 7.6 | — | 20 mL buffer |
| 8 | 100 mM K, pH 7.1 | — | 20 mL buffer |
| 9 | 100 mM K, pH 6.6 | — | 20 mL buffer |
| 10 | 100 mM K, pH 6.1 | — | 20 mL buffer |
| 11 | 100 mM K, pH 5.5 | — | 20 mL buffer |
| 12 | 100 mM K, pH 5.0 | — | 20 mL buffer |
| 13 | 60 mM K, pH 6.1 | — | 20 mL buffer |
| 14 | 30 mM K, pH 6.2 | — | 20 mL buffer |
| 15 | 10 mM K, pH 6.3 | — | 20 mL buffer |
| 16 | 60 mM Na, pH 6.1 | — | 20 mL buffer |
| 17 | 100 mM Na, pH 6.0 | — | 20 mL buffer |
| 18 | 100 mM Na, pH 6.0 | 25% glucose | 15 mL buffer, 5 gram glucose |
| 19 | 100 mM Na, pH 6.0 | 25% glucose, 5% GSH | 14 mL buffer, 5 g gluc, 1 g GSH |
| 20 | 100 mM Na, pH 6.0 | 25% lactose | 15 mL buffer, 5 gram lactose |
| 21 | 100 mM Na, pH 6.0 | 25% trehalose | 15 mL buffer, 5 gram trehalose |
| 22 | 100 mM K, pH 5.3 | 25% glucose | 15 mL buffer, 5 gram glucose |
| 23 | 100 mM K, pH 5.3 | 25% Oligo5-18 | 15 mL buffer, 5 gram oligo |
| 24 | 100 mM K, pH 5.3 | 25% Oligo2-9 | 15 mL buffer, 5 gram oligo |
| 25 | 100 mM K, pH 6.0 | 25% Oligo2-9 | 15 mL buffer, 5 gram oligo |
| 26 | 100 mM K, pH 6.0 | 40% Oligo2-9 | 12 mL buffer, 8 gram oligo |
| 27 | 100 mM K, pH 6.0 | 50% Oligo2-9 | 10 mL buffer, 10 gram oligo |
| 28 | 100 mM K, pH 6.0 | 60% Oligo2-9 | 8 mL buffer, 12 gram oligo |
| 29 | 100 mM K, pH 6.0 | 2% Oligo2-9 | 19.6 mL buffer, 0.4 g oligo |
| 30 | 100 mM K, pH 6.0 | 5% Oligo2-9 | 19 mL buffer, 1 g oligo |
| 31 | 100 mM K, pH 6.0 | 10% Oligo2-9 | 18 mL buffer, 2 g oligo |
| 32 | 100 mM K. pH 6.0 | 25% Oligo2-9, 5% GSH | 15 mL buffer, 5 g oligo |
| 33 | 100 mM K, pH 6.1 | 20% Oligo2-9 | 16 mL buffer, 4 g oligo |
| 34 | 100 mM K, pH 6.1 | 30% Oligo2-9 | 14 mL buffer, 6 g oligo |
| 35 | 100 mM K, pH 6.1 | 35% Oligo2-9 | 13 mL buffer, 7 g oligo |
| 35 | 100 mM K, pH 6.1 | 25% fructose | 15 mL buffer, 5 g fructose |

*Oligo2-9 is fractionated refined inulin with a degree of polymerisation in the range of 2-9; Oligo5-18 is refined inulin with a degree of polymerisation in the range of 5-18

### Example 2—Conventional Storage Solutions do not Preserve Hydrolase Activity

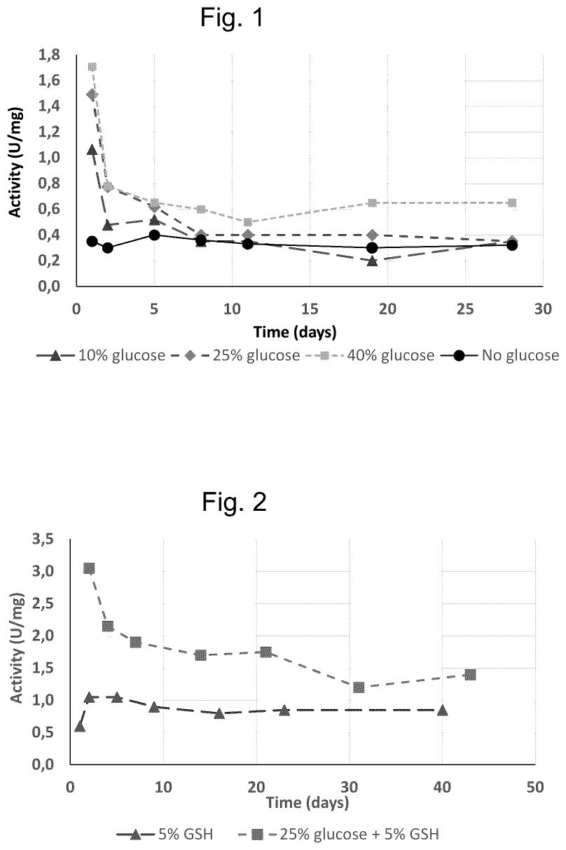

Enzymes are often stored in the presence of glucose or of antioxidants such as glutathione (GSH). FIG. 1 shows that this does not preserve the activity of hydrolase enzymes to a great extent. Here, model enzyme urease was immobilised and then stored after having been suspended in a storage solution, filtered, and dried. The storage solution contained the indicated amount by weight of glucose, at a pH of 6 to 7. Storage was at 20° C., and enzyme activity was assayed at the indicated moments in time. The presence of a reductant in the solution did not usefully mitigate this loss of activity, as shown in FIG. 2. Glutathione (GSH) was added to a glucose storage buffer, but a loss of almost 60% of enzyme activity occurred within 30 days.

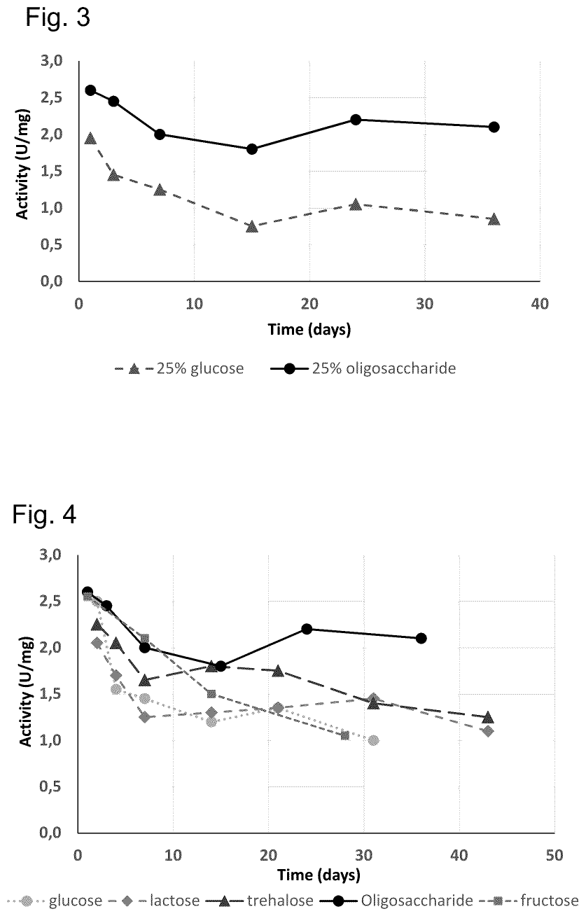

The current golden standard for the storage of Jack Bean urease is storage in sodium phosphate buffer at pH5 in the presence of 25 to 85 wt.-% glucose or lactose. Use according to the invention, wherein a similar amount of oligosaccharide is used instead of glucose, resulted in a preservation of up to 80% urease activity, with activity still remaining well above levels observed for glucose storage even after 120 days. A persisting residual activity of about 1.5 U/mg was observed. A similar result was obtained when potassium phosphate buffer at pH 5 was used instead. Results are shown in FIG. 3.

In conclusion conventional storage solutions are not effective for hydrolase enzymes, while the use of oligosaccharides according to the invention does increase shelf life.

### Example 3—Amount and Type of Carbohydrate

Various sugars were screened for their effect on urease stabilisation. Monosaccharides (glucose, fructose), disaccharides (trehalose, fructose), and oligosaccharides (fractionated refined inulin with a degree of polymerisation in the range of 2-9) were tested. as shown in FIG. 4. A convincing difference is when fructose is compared to the oligofructose. In the presence of the monosaccharide a continues decline of enzyme activity is observed to less than half of the initial activity within 30 days of storage, the loss of activity over time is stabilized up to 80% of the initial activity when these fructose moieties are linked to a linear chain of predominantly 5-8 units as for the oligosaccharide. Fructose eventually does not outperform the monosaccharide glucose. Disaccharides like lactose (a linked galactose and glucose unit) or trehalose (2 glucose units) outperform the monosaccharides, but do not have the same impact on preserving enzyme activity as fractionated refined inulin with a degree of polymerisation in the range of 2-9.

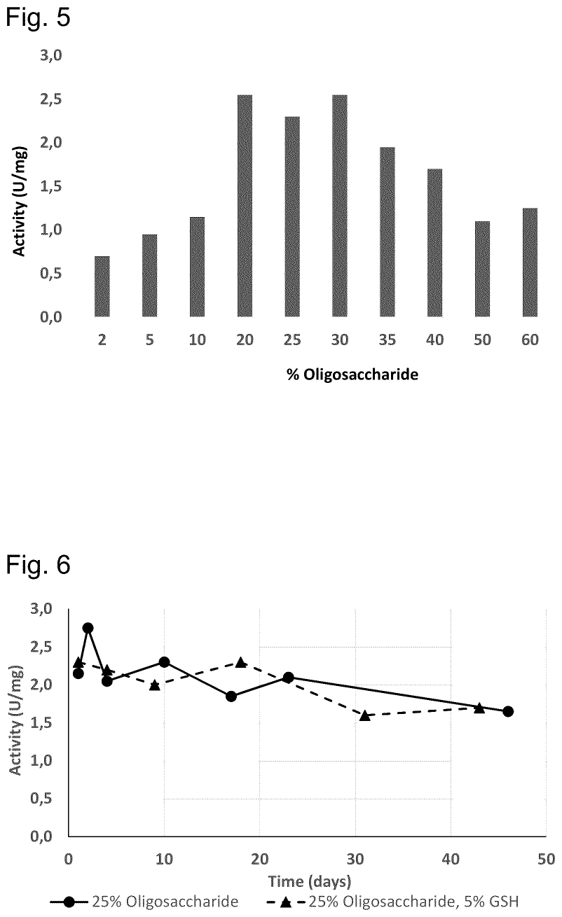

FIG. 5 shows the enzyme activity of Jack Bean urease upon storage at 20° C. in the presence of various amounts of oligosaccharide, ranging from 2 to 60 weight percent. The positive enzyme activity stabilizing effect increases up to about 20% fractionated refined inulin with a degree of polymerisation in the range of 2-9, levels between 20 and 30 weight % and decreases gradually above 30%. This illustrates that there is an optimal range where the stabilizing effect of the presence of oligosaccharide is largest.

### Example 4—the Effect of the Oligosaccharide is Dominant

As was shown in FIG. 2 the presence of 5% of glutathione (GSH) improves the stability of enzyme activity when stored in a 25% glucose-solution, leading to an activity that is about 30% higher after 40 days of storage. However, this activity was still lower than when oligosaccharides were used without additional reducing agent. FIG. 6 demonstrates that additional reducing agents have no clear effect on the preserved enzyme activity when combined with oligosaccharides.

### Example 5—Terminal Glucose Residues Improve Enzyme Stability

Immobilized urease was suspended in four different solutions, after which the suspensions were filtered and the residue was freeze dried and assayed for urease activity. All solutions in this example were 23 wt.-% solutions comprising monodisperse saccharides with a degree of polymerisation of 2. Urease activity was highest for the compound comprising two terminal glucose residues (trehalose). Results are shown in table 5.

TABLE 5
Urease activity after
freeze drying (U/mg)

| Stabilising | Activity |
|---|---|
| agent | (U/mg) |
| Trehalose | 3.1 |
| Maltose | 2.2 |
| Sucrose | 2.3 |
| Lactose | 1.4 |

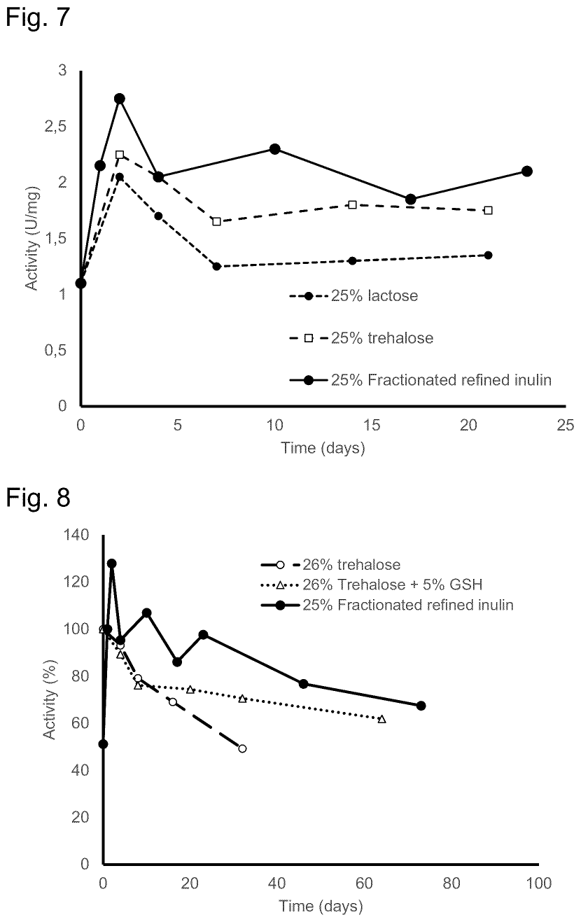

This stability was found to persist over time. In an additional experiment, compositions of 100 mM phosphate buffer at pH 6 and 25 wt.-% stabilising agent were used for suspending immobilized urease. It was then filtered and dried. Trehalose provided more stability than lactose. Fructooligosaccharide was most effective. Results are shown in FIG. 7.

### Example 6—Fructooligosaccharide Outperforms Disaccharides

Immobilized urease was suspended in three different solutions, after which the suspensions were filtered and the residues were dried and assayed for urease activity. Solutions were 26 wt.-% solutions when comprising disaccharides, or 25 wt.-% solutions for fructooligosaccharide. Urease activity was highest for the fructooligosaccharide. The stabilising effect of trehalose could be further improved by addition of an antioxidant (here glutathione), which is surprising in light of the lack of effect that GSH was found to have for longer chained compounds (see Example 4). Results are shown in FIG. 8. Fructooligosaccharide maintained enzyme activity at almost 80% after almost 80 days. Trehalose with GSH maintained about 60% after about 60 days, while trehalose without GSH dropped well below 60% within about 30 days.

1. Method for improving the stability of an enzyme, comprising the steps of:
i) providing an enzyme;
ii) contacting the enzyme with a storage solution comprising an oligosaccharide to obtain a storage composition, and
iii) optionally drying the storage composition.

2. The method according to claim 1, wherein the enzyme is a hydrolase.

3. The method according to claim 1, wherein the enzyme has an active site comprising a nickel center.

4. The method according to claim 1, wherein the storage solution further comprises buffer salts, antioxidants, bacteriostatics, chelators, cryo-protective agents, or serum albumins.

5. The method according to claim 1, wherein the storage solution is buffered at a pH in the range of 5.5-8.2, and/or wherein the storage solution is a pharmaceutically acceptable solution.

6. The method according to claim 1, wherein the storage solution comprises 5-40 wt.-% of the oligosaccharide.

7. The method according to claim 1, wherein the storage solution comprises about 25 wt.-% of the oligosaccharide and optionally buffer salts.

8. The method according to claim 1, wherein the oligosaccharide is an oligohexose.

9. The method according to claim 1, wherein the oligosaccharide has a degree of polymerisation of 2-75.

10. The method according to claim 1, wherein the enzyme is an immobilized enzyme.

11. The method according to claim 1, wherein the storage composition is stored for at least 25 days, wherein the enzyme retains at least 75% of its original activity after the storage.

12. Composition comprising an hydrolase and an oligohexose.

13. The composition according to claim 12, wherein the enzyme is an amidohydrolase, or wherein the oligosaccharide is an oligohexose.

14. The composition according to claim 12, wherein the composition is comprised in a cartridge.

15. Method for storing an enzyme, the method comprising the steps of:
I) providing a composition according to claim 12, and
II) storing the composition for at least 2 days.
